(19) United States
(12) Patent Application Publication
(10) Pub. No.: US 2023/0417889 A1
(43) Pub. Date: Dec. 28, 2023

(54) METHODS FOR INVERSION OF SENSORS AND EMITTERS ORIENTATION IN MULTI-SENSOR IMAGING

(71) Applicant: Ikko Health Ltd., Tel Aviv (IL)

(72) Inventors: Ramon AXELROD, Kiryat Ono (IL); Amihai VIKS, Ramat-Gan (IL); Moshe OZIEL, Tekoa (IL); Edan GOFER, Katy, TX (US)

(73) Assignee: Ikko Health Ltd., Tel Aviv (IL)

(21) Appl. No.: 18/342,026

(22) Filed: Jun. 27, 2023

Related U.S. Application Data
(60) Provisional application No. 63/367,161, filed on Jun. 28, 2022.

(51) Int. Cl. G01S 7/52

(52) U.S. Cl. CPC G01S 7/5205; A61B 8/4245

(57) ABSTRACT

Techniques for orientation determination cause emitters of a multi-sensor imaging apparatus to emit a first set of ultrasound signals; measuring arrival times and amplitudes of the first set of ultrasound signals; estimating initial locations of the sensors; pointing each of the sensors towards a center mass location; fitting an initial orientation to each of the sensors and to each of the emitters using a loss function; determining a coarse model based on the initial locations of the sensors and initial locations of the emitters; calculating a new location and a new orientation for each of the sensors and each of the emitters based on the coarse model and a second set of ultrasound signals; employing a full wave inversion to generate an updated model; and determining an orientation of each of the sensors and emitters based on the updated model. Embodiments may include filtering out outlier sensor-emitter pairs.

### CROSS-REFERENCE TO RELATED APPLICATIONS

This application claims the benefit of U.S. Provisional Application No. 63/367,161 filed on Jun. 28, 2022, the contents of which are hereby incorporated by reference.

### TECHNICAL FIELD

The present disclosure generally relates to ultrasound imaging systems, and more particularly to determination of location and orientation of ultrasound emitters and sensors of an ultrasound imaging system.

### BACKGROUND

Ultrasound is commonly used for a variety of applications including that of scanning body parts non-invasively and at low-risk. In a typical application, an array of ultrasound sensors is mounted on a flat surface. The body part to be scanned is coated with a gel to ensure better matching between the surface of the sensors and the skin of the body part. The array emits a tight beam that is used to sweep the outer surface of a volume of interest and produce images of the inner volume from the returned signals. One or more emitters emit ultrasound waves in a desired frequency and the reflected or refracted sound waves are captured by the array of sensors. The signals are then interpreted to provide an image of the internal organs and bones of the body part. In a typical setting, a patient is placed in a desired position to enable the use of the array of ultrasound sensors by a practitioner. Over time, ultrasound imaging capabilities have improved and from images that only expert interpreters could decipher, it is possible today to provide three-dimensional imaging, which is evidently valuable, for example, when scanning a fetus in the womb with 3D details.

A drawback of currently implemented ultrasound devices using an array of sensors/emitters is that the array is typically small in surface area and, therefore, when larger areas of the body need to be checked, the ultrasound measuring devices must be manually or automatically moved along the patient's skin in order to reach the required coverage. Moreover, since the devices rely on beamforming, an adequate contact between the ultrasound array of sensors and the patient's skin must be maintained throughout the whole process for effective and indisputable imaging results. That is, ultrasound used in medical imaging is performed using a single emitter/sensor array mounted on a flat surface much larger than a wavelength. The array emits a tight beam that is used to sweep the outer surface of a volume of interest and produce images of the inner volume from the returned signals.

An alternative scanning setup may utilize a set of individual emitters and sensors, spread around the volume of interest at fixed locations. Ultrasound waves emitted by a given emitter propagate through the medium (e.g., a patient's body) and are recorded by all the encompassing sensors. Each sensor records data from multiple emission directions and captures not only the returned signal, but the initially emitted waves as well. A similar emitter/sensor setup is being used predominantly in geoscience applications to explore the Earth's subsurface structure and properties. Generally, inversion methods are applied to infer the Earth's interior properties that best fit the recorded data. In such applications, the position of emitters and sensors on the acquisition surface is a priori known with high certainty. To this end, use of such similar emitter/sensor setup is limited for industrial and medical applications where a direct and accurate measurement of position in real time is not readily obtainable.

In addition, in many medical applications, the sensor size is larger than the ultrasound half wavelength and, thus, determining the sensors' orientations alongside their positions in space may be necessary. Current methods may simultaneously locate the emitters and sensors position while optimizing the medium's inner properties model. However, methods to determine a sensor's orientation on the acquisition surface while optimizing the medium's inner properties model simultaneously are yet to be developed and implemented.

It would therefore be advantageous to provide solutions that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for orientation determination. The method comprises: causing emitters of a multi-sensor imaging apparatus to emit a first set of ultrasound signals, wherein the multi-sensor imaging apparatus includes the emitters and sensors; measuring arrival times and amplitudes of the first set of ultrasound signals; estimating initial locations of the sensors based on the measured arrival times and amplitudes; pointing each of the sensors towards a center mass location; fitting an initial orientation to each of the sensors and to each of the emitters using a loss function; determining a coarse model based on the initial locations of the sensors and initial locations of the emitters; calculating a new location and a new orientation for each of the sensors and each of the emitters based on the coarse model and a second set of ultrasound signals; employing a full wave inversion (FWI) to generate an updated model based on the new locations and the new orientations of the sensors and emitters; and determining an orientation of each of the sensors and each of the emitters based on the updated model.

Certain embodiments disclosed herein also include a non-transitory computer readable medium having stored thereon causing a processing circuitry to execute a process, the process comprising: causing emitters of a multi-sensor imaging apparatus to emit a first set of ultrasound signals, wherein the multi-sensor imaging apparatus includes the emitters and sensors; measuring arrival times and amplitudes of the first set of ultrasound signals; estimating initial locations of the sensors based on the measured arrival times and amplitudes; pointing each of the sensors towards a center mass location; fitting an initial orientation to each of the sensors and to each of the emitters using a loss function; determining a coarse model based on the initial locations of the sensors and initial locations of the emitters; calculating a new location and a new orientation for each of the sensors and each of the emitters based on the coarse model and a second set of ultrasound signals; employing a full wave inversion (FWI) to generate an updated model based on the new locations and the new orientations of the sensors and emitters; and determining an orientation of each of the sensors and each of the emitters based on the updated model.

Certain embodiments disclosed herein also include a system for orientation determination. The system comprises: a processing circuitry; a plurality of emitters communicatively connected to the processing circuitry; a plurality of sensors communicatively connected to the processing circuitry; and a memory, the memory containing instructions that, when executed by the processing circuitry, configure the system to: cause emitters of a multi-sensor imaging apparatus to emit a first set of ultrasound signals, wherein the multi-sensor imaging apparatus includes the emitters and sensors; measure arrival times and amplitudes of the first set of ultrasound signals; estimate initial locations of the sensors based on the measured arrival times and amplitudes; point each of the sensors towards a center mass location; fit an initial orientation to each of the sensors and to each of the emitters using a loss function; determine a coarse model based on the initial locations of the sensors and initial locations of the emitters; calculate a new location and a new orientation for each of the sensors and each of the emitters based on the coarse model and a second set of ultrasound signals; employ a full wave inversion (FWI) to generate an updated model based on the new locations and the new orientations of the sensors and emitters; and determine an orientation of each of the sensors and each of the emitters based on the updated model.

Certain embodiments disclosed herein include the method, non-transitory computer readable medium, or system noted above or below, wherein the at least one outlier sensor-emitter pair is filtered based on signal intensity, wherein each outlier sensor-emitter pair has a signal intensity below a threshold.

Certain embodiments disclosed herein include the method, non-transitory computer readable medium, or system noted above or below, wherein the at least one outlier sensor-emitter pair is filtered based on a characteristic of signals passing through a non-soft tissue that is larger than a predetermined threshold size.

Certain embodiments disclosed herein include the method, non-transitory computer readable medium, or system noted above or below, further including or being configured to perform the following steps: performing a gradient descent.

Certain embodiments disclosed herein include the method, non-transitory computer readable medium, or system noted above or below, wherein the gradient descent is any of: stochastic gradient descent (SGD), Broyden-Fletcher-Goldfarb-Shanoo, limited-memory Broyden-Fletcher-Goldfarb-Shanoo, adaptive moment estimation (ADAM), ADAM-W, multistage stochastic variational approximation gradient (M-SVAG), and ADAbelief.

Certain embodiments disclosed herein include the method, non-transitory computer readable medium, or system noted above or below, further including or being configured to perform the following steps: filtering out at least one outlier sensor-emitter pair based on the estimated initial locations of the sensors.

Certain embodiments disclosed herein include the method, non-transitory computer readable medium, or system noted above or below, further including or being configured to perform the following steps: sorting the sensors based on a loss value determined for each sensor using the loss function; selecting at least one sensor of the sensors for which the determined loss value is above a predetermined threshold value; changing a direction of each of the selected at least one sensor to a direction of maximal intensity; and repeating a process of the sorting the sensors, the selecting at least one sensor, and the changing direction of each selected sensor until a loss value of each sensor is below the predetermined threshold value.

Certain embodiments disclosed herein include the method, non-transitory computer readable medium, or system noted above or below, further including or being configured to perform the following steps: using an inversion tomography.

Certain embodiments disclosed herein include the method, non-transitory computer readable medium, or system noted above or below, wherein the FWI is employed and the orientation is determined iteratively until a model generated based on the orientation converges.

Certain embodiments disclosed herein include the method, non-transitory computer readable medium, or system noted above or below, further including or being configured to perform the following steps: determining a loss value for each sensor based on a full amplitude over all frequencies comparison between an observed measurement and a calculated signal for the sensor.

Certain embodiments disclosed herein include the method, non-transitory computer readable medium, or system noted above or below, further including or being configured to perform the following steps: determining a loss value for each sensor based on a ratio of amplitudes at different frequencies.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a schematic diagram of an ultrasound sensing garment (USG) disposed with sensors and emitters according to an embodiment.

FIG. 2 is a flowchart of model optimization according to an embodiment.

FIG. 3 is a flowchart for determination of orientations of sensors and emitters according to an embodiment.

FIG. 4 is an electronic circuit adapted to implement model inversion of locations and orientations of sensors and emitters in a multi-sensor imaging apparatus according to an embodiment.

FIG. 5A is a pattern in polar coordinates of emitter radiation pattern and sensor receiving pattern according to an example embodiment.

FIG. 5B is a pattern in Cartesian coordinates of emitter radiation pattern and sensor receiving pattern according to an example embodiment.

FIG. 6 is a geometry emission function and orientations according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

The various disclosed embodiments provide systems and methods for improved determinations of orientations of sensors and emitters in a multi-sensor imaging apparatus by implementing stages of preparation, initial orientation, and improvement. In an embodiment, the preparation stage includes measuring arrival times and amplitudes of signals as well as estimating sensors' locations; filtering outlier sensor-emitter pairs; and, as an initial state, pointing each sensor toward the center mass location of the sensors at a sensor's height level.

In an embodiment, the initial orientation stage includes: using gradient based optimization to fit the initial orientations based on a loss function; and, optionally, adding a global maximization comprising: sorting the sensors based on the loss value; selecting the highest loss's sensor and changing the direction of the maximal intensity; and, measuring the loss, if loss improved return to the initial orientation stage; otherwise, continue with the improvement stage that includes: recalculating the location and orientation of sensors and emitters; and, repeating improvement of the model based on the new location and orientation.

The disclosed embodiments include, but are not limited to, a system and method which provide or utilize techniques for solving the problem of inversion of orientation of ultrasound sensors and ultrasound emitters of a multi-sensor imaging apparatus. This allows for ensuring accurate ultrasound image representation when the orientation of both emitters and sensors change with respect to each other, for example, as would be the case when such emitters and sensors are attached to an ultrasound sensing garment.

While it is recognized that some solutions could hypothetically utilize a human operator to mitigate inversion of orientation of ultrasound sensors and ultrasound emitters of a multi-sensor imaging apparatus, such solutions fail in practice. A human operator applies subjective criteria to analyze, determine, and classify, leading to results which are inconsistent between different human operators, and often inconsistent even between the same human performing the same task repeatedly, and in particular at the speeds required to provide an operable solution. Moreover, the number of possibilities of orientation in space of a plurality of emitters and a plurality of sensors by far exceeds any practical use of the human mind.

Various disclosed embodiments utilize a set of predefined objective criteria, such as measuring arrival time of the ultrasound signals and amplitude thereof, estimating the sensors' locations based on the measured arrival times and amplitudes, filtering out outlier sensor-emitter pairs, pointing each of the sensors towards a center mass location, fitting an initial orientation to each of the sensors and the emitters using a loss function, determining a coarse model based on the initial location of the sensors and emitters, recalculating a new location and a new orientation of each of the sensors and the emitters, employing full wave inversion (FWI) to generate an improved model based on the new locations and the new orientations of the sensors and emitters, and improving the orientation of the sensors and the emitters based on the improved model. This use of such objective criteria results in solving the problem of inversion of orientation of ultrasound sensors and ultrasound emitters of a multi-sensor imaging apparatus in a reliable and consistent manner based on the objective criteria.

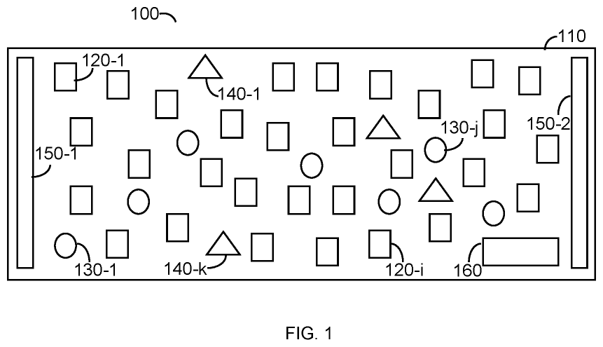

FIG. 1 depicts an example schematic diagram 100 of an ultrasound sensing garment (USG) 110 disposed with sensors 120, emitters 130, and markers 140 embedded therein according to an example embodiment. Such a USG is described, for example but not by way of limitation, in PCT patent application no. PCT/162021/061474, titled “Wearable Garment Adapted for Ultrasound Sensing and Methods Thereof for Full Wave Inversion with Imprecise Sensor Positions” and assigned to common assignee the contents of which are hereby incorporated by reference.

Embedding of the sensors 120, emitters 130, and markers 140 may be achieved by different techniques such as, but not limited to, weaving, gluing, mechanically attaching, and the like, and any combination thereof. The USG 110 is designed to provide an imaging solution that transmits and receives ultrasound signals that are processed to generate a high-resolution, three-dimensional (3D) image of a scanned body part. In an embodiment, a power supply (not shown) may be provided to elements embedded into the USG 110 (e.g., elements such as the sensors 120, the emitters 130, the markers 140), which may be provided by a mesh of conducting wires (also not shown) that are part of the USG 110.

It should be further appreciated that the USG 110 may be shaped in various ways to be worn on a body part or parts, or otherwise wrapped thereabout. In an embodiment, the USG 110 is designed to be laid upon a body part rather than be worn on the body part or wrapped around the body part. In another embodiment, one or more of the elements embedded into the USG 110 (i.e., the sensors 120, emitters 130, the markers 140, or a combination thereof) may be coated with a soft, polymer-based material which allows for sufficient contact, essentially without air gaps, between the element and the body part it is adjacent to. In yet another embodiment, the garment material is elastic and adapted to tightly correspond to the contour of the body part.

The USG 110 is further designed to comfortably fit about a body part having the necessary flexibility otherwise not provided by at least some existing solutions. Such flexibility is achieved, at least in part, by avoiding the need for sensor arrays that are typically large and bulky. Such sensor arrays may cause the wearable device to have a rigid feeling.

In order to facilitate providing a flexible garment 110, in an embodiment, miniature ultrasound sensors 120, for example, sensors 120-1 through 120-i, where ‘i’ is an integer greater than ‘1’(the ultrasound sensors 120 also referred to herein simply as sensors 120 or as a sensor 120 for simplicity), are configured on the USG 110. The sensors 120 may be embedded randomly or orderly within the USG 110. In a further embodiment, the sensors 120 are each small in size relative to the garment 110 such that, unlike an implementation utilizing an array of sensors, the garment 110 retains its flexibility in such an embodiment.

In an embodiment, such sensors 120 do not abut each other, and each sensor 120 may be positioned upon the USG 110 as may be deemed necessary or desired for a given use case, maintaining at least a predetermined distance from one another. The plurality of sensors 120 on the USG 110 may be referred to as a loose array because each sensor 120 is separate from any neighboring sensor 120. The sensors 120 may be, for example but not limited to, piezo-electric sensors, capacitive micro electrical Mechanical Systems (MEMS) based sensors, capacitive polymer-based sensors, combinations thereof, and the like. In addition to the sensors 120, the USG 110 also includes ultrasound emitters 130, referred to herein also as emitters 130, for example emitter 130-1 through 130-j, where T is an integer greater than ‘1’.

The USG 110 further includes markers 140, for example markers 140-1 through 140-k, where ‘k’ is an integer greater than or equal to ‘1’. The markers 140 may be used to obtain initial approximate positions of the emitters 130 and sensors 120. The initial approximation of positions of emitters and sensors are discussed in greater detail herein. Power supply to the USG 110 may be provided using a variety of sources such as, but not limited to, batteries, a generator, a power outlet, and the like.

In an embodiment, the USG 110 may be further configured with fasteners 150, for example fasteners 150-1 and 150-2, adapted to secure the USG 110 around a body part. The fasteners 150 may include, but are not limited to, hooks, Velcro®, buttons, and corresponding button loops or holes, combinations thereof, portions thereof, and the like.

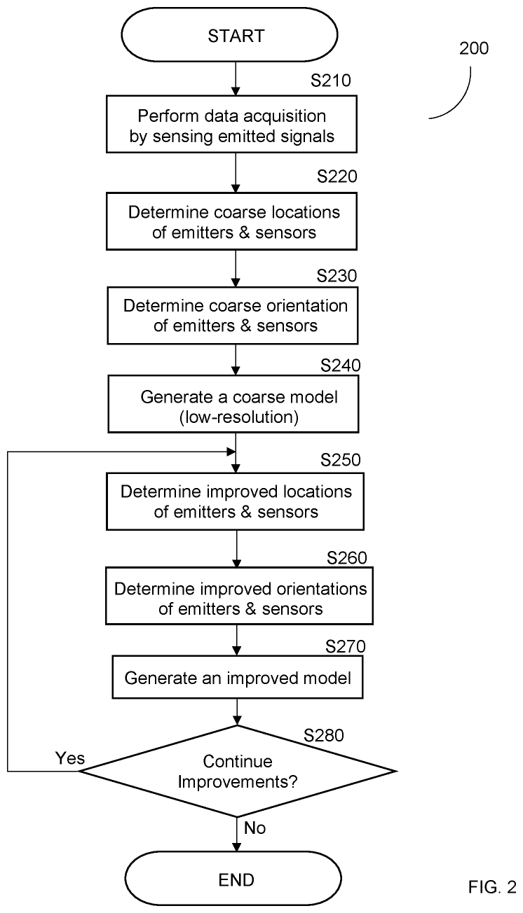

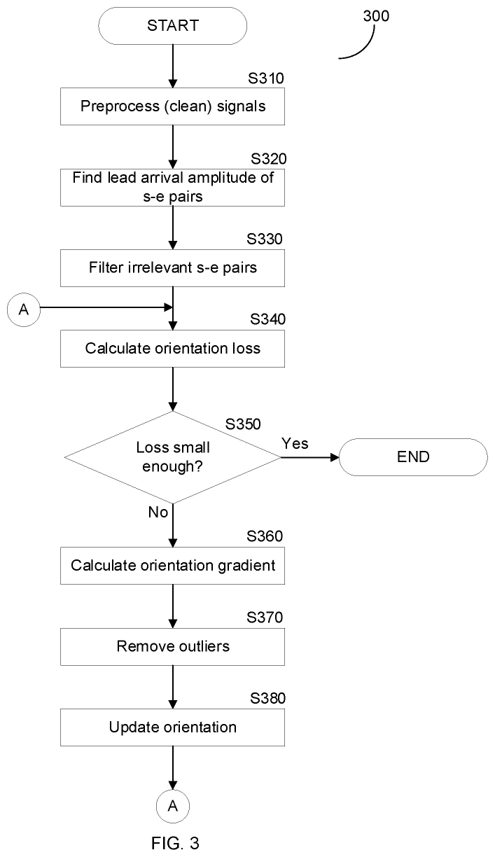

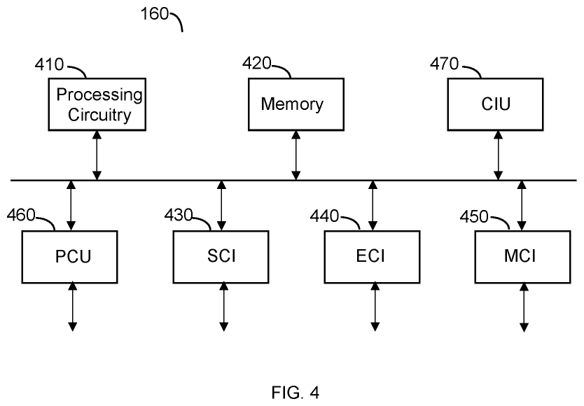

In an embodiment, the USG 110 may be equipped with an electronic circuit 160 that is adapted to provide the power for consumption by the elements (e.g., the sensors 120, emitters 130, the markers 140, or a combination thereof) embedded into the USG 110. The electronic circuit 160 may include, but is not limited to, a combination of digital, analog, and optical components as may be adapted to allow for the proper operation of the USG 110. The signals received from the sensors 120 may be processed by the electronic circuit 160, as further detailed below with respect to FIG. 4, either locally or at a processing device (not shown) in order to further process the signal and to display, on a display device (not shown), an image corresponding to the processed signals. In an example embodiment, the signals are transmitted to the processing device by, for example but not limited to, a wired or a wireless connection after initial or minimal processing. The processing of the signals is described further with respect to FIGS. 2 and 3 herein.

In an embodiment, at least a portion of a process performed via the electronic circuit 160 or otherwise via computing components of or communicating with the USG 110 includes: a. inverting for the elastic properties of the medium (e.g., full waveform inversion); and b) obtaining the location of one or more elements (e.g., one or more of the sensors 120, one or more of the emitters 130, or both).

To this end, in an embodiment, such computing components may be configured to calculate, from a set of observed signals, a causal factor that produces them. It is the inverse of the forward problem which starts with causes and computes the results. The inversion is performed in order to attempt to find the best model that fits the acquired data. Herein, the term “full wave form inversion” (FWI) refers to, given some initial model, a method that simulates waves from the emitters (for example, the emitters 130) to the sensors (for example the sensors 120) and compares the measured signals. It should be further appreciated that reference to FWI is interchangeable with any of its variants, including, but not limited to, Adaptive wave form inversion (Awl), wavefield reconstruction inversion (WRI), and other like algorithms.

Errors between the simulated waves and the measured signals are back propagated to obtain a gradient for each point in the model and the model is changed (in some implementations, only slightly, i.e., less than a threshold amount or proportion) in the gradient direction, thereby resulting in a new model. The new model is then used instead of the previous model to simulate the waves, forwards and backwards, and to recompute the gradient. In an embodiment, iterations of changing the model based on simulation, measurement, and comparison are continued until the model converges. In various implementations, the model is considered to be close to the “true” model, or otherwise sufficiently close for all practical matters once converged.

In some implementations, the locations of the sensors are identified along with the model using a process which begins by assuming a very simple model and compares the simulated time travel of waves through the medium to the actual travel time. The difference in travel time is used to correct the locations of the emitters, for example the emitters 130. The difference in travel time in various soft tissue is such that even high uncertainty in human body models achieves a good enough initial location error. Using the initial location, it is possible to build a better model and therefore to improve the location identification. This alternating process continues until both the model expected locations (e.g., locations of sensors as indicated in the model) and actual location of the sensors (e.g., actual locations of the sensors determined based on the current measurements) sufficiently converge.

It should be noted that some initial care is needed to deal with non-soft tissue (e.g., bones, air, etc.) in the model. Though some differences in attenuation of close frequency signals cause a large enough attenuation as to be virtually irrelevant, some differences in attenuation of close frequency signals may need careful consideration in the model in at least some implementations.

FIG. 2 is an example flowchart 200 illustrating a method of model optimization according to an embodiment. The method described herein may utilize a loose array of ultrasonic emitters (for example, the emitters 130, FIG. 1) and sensors (for example, the sensors 120, FIG. 1) that are spread around a body in unknow or otherwise potentially inaccurately known geometry. It should be noted that FIG. 2 is described with respect to sensors 120 and emitters 130 of the USG 110, FIG. 1, but that at least some of the disclosed embodiments utilizing the process of FIG. 2 are not limited to the particular configuration of the USG 110 illustrated in FIG. 1.

The process begins with a data acquisition phase at S210, in which each emitter of the emitters 130 emits a known radiation pattern. The emission can be done sequentially (i.e., each emitter in its turn) or in parallel (i.e., simultaneously) using, for example, but not limited to, encoding. During the emission phase, signal arrival data is recorded by each sensor of sensors 120 for all sensors of the USG 110, FIG. 1. After obtaining raw signals, a signal processing may be used to remove noise and artifacts.

Based on data acquired at S210, locations and orientations of elements such as emitters and sensors may be determined iteratively. The location of an element such as an emitter or sensor refers to a spatial position of that element with respect to a medium (e.g., a body part), and the orientation of such an element refers to an angular direction in which the element points to in its position. For example, a first emitter may be placed in location A, above the belly button, with an orientation that points the emitter along the surface of the skin. In the same example, the first emitter may be placed in location A with an orientation that points the emitter to emit into the tissue below, perpendicular to the skin surface.

In an embodiment, each of the coarse locations and the coarse orientations may be determined based on the data acquired at S210 and further based on an initial model. The initial model may be based on a predetermined configuration of the model, for example, based on known locations and orientations of the model at a default or starting position.

At S220, coarse locations of the emitters 130 and the sensors 120 are determined using the coarse model generated at S220. In an embodiment, each coarse location is determined based on the delay between the time at which the emitters 130 sent a signal and the time at which the signal was received by the sensors 120. Each such delay forms a constraint on the distance between the emitter and the sensor, or more precisely, a distance range. All the delays form a network of constraints which may be solved to find the corresponding locations.

At S230, coarse orientations of the emitters 130 and the sensors 120 are determined using the coarse model generated at S220. In an embodiment, in order to determine the orientation of emitters 130 and the orientation of sensors 120, it is assumed that the emission pattern of the sensors 120 and emitters 130 is known in advance and that the sensors 120 are spread so that actual intensities for multiple angles may be measured. These measurements can be “fitted” to the known emission pattern in order to determine the orientation. In practice, the sensors 120 themselves also have an orientation and sensitivity pattern for which details of handling are provided herein. In some embodiments, the coarse orientations may be determined as described further below with respect to FIG. 3.

At S240, a coarse model is generated based on the coarse locations and orientations. To this end, given the coarse location as determined in S220 and the coarse orientation as determined in S230, a coarse model can be computed using techniques such as, for example but not limited to, by using full waveform inversion techniques. This coarse model may, in turn, be used to determine a new improved location in S250 and a new improved orientation in S260 for each of the emitters 130 and sensors 120. In some embodiments, the improved orientations may be determined as described further below with respect to FIG. 3. Thereafter, at S270, an improved model (or updated model) is generated, for example, but not limited to, by making use of FWI techniques.

At S280, it is checked whether additional iterations are to be performed and, if so, execution continues with S250; otherwise, execution terminates. In an embodiment, the improved model (S270) is applied to modify the orientation of the sensors and the emitters for accurate ultrasound imaging using the multi-sensor imaging apparatus as disclosed herein. The implementation is therefore provided in two phases, a coarse phase (S220, S230, and S240) and a repetitive improvement phase (S250, S260, and S270). During the repetitive improvement phase, as the model approximation and location are improved, the orientation is also recalculated. It should be noted that the two-phase implementations allows for optimization of the orientations of sensors and emitters while simultaneously optimizing the model for accurate and improved fitting of ultrasound signals.

FIG. 3 is an example flowchart 300 illustrating a method for determining orientations of sensors and emitters according to an embodiment. In some embodiments, the method of FIG. 3 may be utilized during step S230, step S260, or both, FIG. 2.

It should be noted that FIG. 3 is described with respect to sensors 120 and emitters 130 of the USG 110, FIG. 1, but that at least some of the disclosed embodiments utilizing the process of FIG. 3 are not limited to the particular configuration of the USG 110 illustrated in FIG. 1.

At S310, a set of signals is acquired at the sensors 120 from the emissions of emitters 130 and preprocessed. The received signals are preprocessed at least to clean noise and certain artifacts.

At S320, a lead (first) arrival time and an intensity of the signal are determined for each sensor-emitter pair, for example, the sensor 120-1 and the emitter 130-1, also referred to herein as an s-e pair. The lead arrival signal is determined using cross correlation. Based on predetermined locations of the emitters 130 and the sensors 120 (e.g., locations indicated in a model of a USG or other garment including the sensors and the emitters 130 being used for the current iteration of orientation determination such as an initial or default model, the coarse model determined at S240, or the improved model generated at S270), the intensity of a received leading signal recorded by each sensor 120 from each emitter 130 may act as a set of constraints on the orientation of the sensors 120 and emitters 130, also referred to herein as a transfer matrix.

At optional S330, one or more irrelevant s-e pairs may be filtered out. The irrelevant s-e pairs may be identified by their respective lead arrival time and intensity that are outside of a predetermined range with respect to that of other s-e-pair measurements, with respect to predefined values, or both. In an embodiment, the irrelevant s-e pairs may be, for example but not limited to, s-e pairs relating to a large enough (e.g., above a threshold) attenuation and signal deterioration which may be caused by non-soft tissues such as, but not limited to, bones, air, and the like, and any combination thereof.

At S340, an orientation loss is determined. In an embodiment, a set of orientations is determined via optimization, and the set of orientations is utilized to determine the orientation law. In a further embodiment, given radiation pattern I(θ) of sensors 120 and emitters 130 as a function of the angle from the sensor axis, defined as the orientation vector, and a set of locations and orientations, {pᵢ}, {dᵢ}, the orientation loss is computed based on the difference between actually observed intensity and simulated intensity as defined in Equation 1:

$$Equation 1 :$$

$$J = \sum {( {I}_{s , e}^{calc} - {I}_{s , e}^{obs} )}^{2} = {\sum }_{s , e} {[ \frac{I ( {θ}_{s} ) I ( {θ}_{e} )}{dist ( {p}_{i ,} {p}_{j} )} A ( {p}_{i} , {p}_{j} ) - {I}_{s , e}^{obs} ]}^{2} = {\sum }_{s , e} {[ \frac{I ( {d}_{s} , {p}_{s} - {p}_{e} ) I ( {d}_{e} , {p}_{e} - {p}_{s} )}{dist ( {p}_{i} , {p}_{j} )} A ( {p}_{i} , {p}_{j} ) - {I}_{s , e}^{obs} ]}^{2}$$

In Equation 1, A(x,y) is the attenuation between x,y along a ray trace, and dist(x,y) is the distance along the ray path. In some embodiments, attenuation and distance functions may be replaced with functions of the Euclidean distance when the model resolution is insufficiently low. Thereafter, at an improvement stage (e.g., S250-S270 described with respect to FIG. 2), the ray trace is used to determine the attenuation.

At S350, it is determined if the orientation loss is below a predefined threshold value and, if so, execution terminates; otherwise, execution continues with S360. It should be noted that the orientation loss being small enough (e.g., below a threshold) indicates a sufficient match between the simulated and actually observed signals and thus, the orientations of the sensors and emitters are optimized.

At S360, an orientation gradient is determined. The orientation gradient is a direction of a maximum change in intensity of the signals received at S310. In some implementations, the orientation gradient is determined so as to update orientations in order to minimize the loss (e.g., the loss calculated using the loss function as discussed above with respect to S340). If the initial orientation is close enough to a global minimum of the orientation gradient, this can be solved by a gradient-based optimization method including: a) performing an iterative process that calculates a loss value from the differences between computed (or simulated) and observed signal intensities, and, b) reorienting the emitters 130 and sensors 120 according to the gradient of the loss with respect to an orientation change of each emitter and sensor.

At S370, outliers are removed. Because the model is only roughly known at the coarse stage (see S240), it is possible that the ray trace or signal attenuation for certain s-e pairs, as indicated in the orientation gradient calculated at S360, are different than would be expected based on the model (e.g., different above a predetermined threshold). Such s-e pairs may be identified as outliers and removed. As a non-limiting example, if a given s-e pair with a first emitter (e.g., emitter 130-1) and a first sensor (e.g., sensor 120-1) provides information that is sufficiently different (e.g., above a predetermined threshold) with other s-e pairs of a loose array of ultrasonic emitters and sensors, that given s-e pair is discarded.

In an embodiment, the outlier removal may be weighted with each iteration. The weighting is used to prefer a subset of s-e pairs that are expected to provide better information than other s-e pairs. The weights may be based on prior knowledge (for example, but without limitations, for s-e pairs above the pelvis bone and below the pelvis bone) and signal quality (for example, but without limitations, when the lead signal structure is cleaner, stronger, or both).

At S380, the orientation information for sensors 120 and emitters 130 is updated and execution continues with S340 where a new orientation loss is calculated based on the updated orientation information.

FIG. 4 depicts an example electronic circuit 160 adapted to implement model inversion of locations and orientations of sensors and emitters in a multi-sensor imaging apparatus according to an embodiment. For simplicity and without limitation of the disclosed embodiments, FIG. 4 will also be discussed with reference to elements shown in FIG. 1.

A processing circuitry 410 is communicatively connected to a memory 420. At least a portion of the memory 420 contains therein instructions that, when executed by the processing circuitry 410, enable the USG 110 to perform the functions described herein, and in particular those described in FIGS. 2 and 3 and their respective descriptions. A sensor control interface (SCI) 430, communicatively connected to the PE 410, is adapted to at least receive signals that are sensed by the sensors 120. The SCI 430 may receive signals in parallel from all, part, or just one of the sensors 120. An emitter control interface (ECI) 440, communicatively connected to the PE 410, is adapted to at least send, to the emitters 130, control signals in order to activate the emitters 130. The ECI 440 may transmit signals in parallel to all, part, or just one of the emitters 130.

In an embodiment, an optional marker control interface (MCI) 450, communicatively connected to the PE 410, may be used for active markers 140, and is adapted to at least activate the active markers 140. The MCI 450 may transmit control signals in parallel to all, part, or just one of the active markers 140. A power control unit (PCU) 460, connected to the PE 410, is configured to provide the necessary operational power to any element (e.g., sensors 120, emitters 130, markers 140, or a combination thereof) of the USG 110 which can be performed in parallel, part, or just a single element of the USG 110.

In addition, a communication interface unit (CIU) 470 is communicatively connected to the PE 410. The CIU 470 is configured to provide communications to and from the USG 110. For example, and without limitation, the CIU 470 may be configured to: a) communicate means to activate the USG 110; b) receive signals from an external device (not shown) controlling the USG 110; and c) transmit processed or raw signals captured by the sensors 120 according to any of the embodiments described herein. In one embodiment of the electronic circuit 160, the PE 410 and the memory 420 are replaced by, for example and without limitation, a combinational logic circuitry adapted to perform the tasks discussed herein. Such and similar embodiments are to be considered within the scope of the disclosed embodiments.

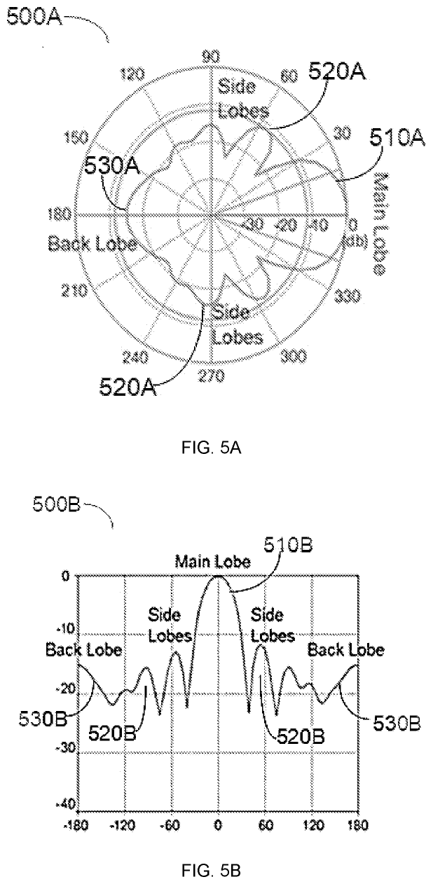

FIG. 5A is an example pattern 500A provided in polar coordinates of an emitter radiation pattern and a sensor receiving pattern according to an embodiment. FIG. 5B is an example pattern 500B provided in Cartesian coordinates of an emitter radiation pattern and a sensor receiving pattern according to an embodiment. Both FIG. 5A and FIG. 5B show the main lobe 510, the side lobes 520, and the back lobe 530, each labeled with “A” and “B” for the respective figures (e.g., 510A for FIGS. 5A and 510B for FIG. 5B).

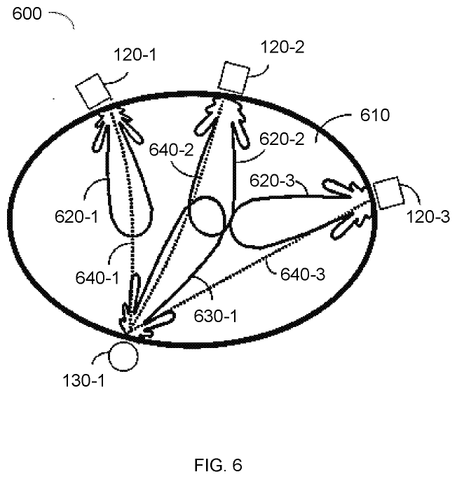

FIG. 6 is an example diagram 600 of a geometry emission function and orientations according to an embodiment. Sensors 120 and an emitter 130 are placed around a body part 610. In the non-limiting example implementation shown in FIG. 6 the sensors include three sensors 120-1 through 120-3.

Reception for each sensor 120 is described along a main reception path 640, for example, the reception path 640-1 is between the emitter 130 and the sensor 120-1. Each emitter has a respective emission lobe, for example the emission lobe 630-1 for the emitter 130-1, with a main lobe and side lobes (main and side lobes not separately labeled). Each sensor has a respective reception lobe, for example the reception lobe 620-2 for the sensor 120-2, with its corresponding main lobe and side lobes. This is of course similar for the sensors 120-1 with reception lobes 620-1, and sensor 120-3 with reception lobes 620-3.

It should be noted that the reception paths 640 are defined based on at least the orientations, the inner properties of the medium (e.g., the body part 610), a combination thereof, and the like. The following emission function and orientations are provided for further understanding of the positioning of the sensors 120 and emitters 130 with respect to the explanations provided herein. It should be noted that FIG. 6 shows one emitter 130 for simplicity and illustrative purposes, and that more than one emitter mat be placed around the body part 610 without departing from the scope of the disclosed embodiments.

In an example embodiment, the positions of the sensors 120 are known based on, for example, time of arrival of the signals. To estimate the orientation of the sensors 120, a calculation based on the measured signal strength is used. For each pair of emitter-sensor (s-e pair), the intensity is defined in Equation 2 as follows:

$${I}_{ij} = \frac{{\sum }_{k = 1}^{N} {{A}_{ij} ( k )}^{2}}{N} \qquad Equation 2 :$$

In Equation 2, i,j are emitter-sensor indices, Aᵢⱼ is the amplitude at k position and N is signal length. The calculated amplitude of the signal for each emitter-sensor pair depends on emitter-sensor mutual orientation and is defined in Equation 3 as follows:

Aᵢⱼ=P(t)*Kₑ(r,θ)*Kₛ(φ)*A Equation 3:

In Equation 3, P(t) is the pressure at time t, Kₑ(r, θ) is the angular kernel function of an emitter 130, r is the emitter-sensor distance, and θ is the emitter-sensor spatial angle, Kₛ is the sensor's 120 angular kernel, φ is the sensor 120 orientation related to the emitter 130, and * is a convolution operator. A is a general operator that defines the other kernels, such as the attenuation over the medium, emitter 130 frequency response and sensor 120 frequency response.

In an embodiment, calculation of the intensity for multiple frequencies uses two or more pulses, each pulse with a different central frequency. In an embodiment (e.g., if the frequencies are not too far apart so that the ray paths are similar [e.g., within a threshold distance of each other]), the measurement of intensity in the transfer matrix may be replaced with a ratio of intensities, i.e., a ratio of amplitudes at different frequencies. The ratio of intensities as a function of angle may provide an improved loss function as it is less dependent on the path itself and certain artifacts and obstructions along the path are easier to remove.

A few non-limiting example loss functions based on a set of intensity-orientation restrictions are defined in Equations 4, 5, and 6 herein. Loss function Equation 4 is as follows:

$$J = \sum {( \frac{{I}_{ij} - {s}_{ij}}{{I}_{ij}} )}^{2} \qquad Equation 4 :$$

In Equation 4, Iᵢⱼ and Sᵢⱼ are the measured and simulated intensities, respectively, of the i,j emitter-sensor pair. For the multiple frequency embodiment, the relevant loss function described in Equation 5 is as follows:

$$J = \sum [ {( \frac{{I}_{ij} ( {f}_{1} )}{{S}_{ij} ( {f}_{1} )} - 1 )}^{2} + {( \frac{{I}_{ij} ( {f}_{2} )}{{S}_{ij} ( {f}_{2} )} - 1 )}^{2} ] \qquad Equation 5 :$$

In Equation 5, f₁ and f₂ are different central frequencies of two different emitted signals. Finally, loss function Equation 6 is as follows:

$$J = \sum {( \frac{{I}_{ij} ( {f}_{1} )}{{S}_{ij} ( {f}_{1} )} - \frac{{I}_{ij} {f}_{2} )}{{S}_{ij} ( {f}_{2} )} )}^{2} \qquad Equation 6 :$$

In yet another embodiment, an additional term is added to the loss function (Equation 5). It is used so as to avoid high curvature of the surface formed by connecting all surfaces assumed to lie on the surface of the organs.

According to an embodiment, the method of model optimization includes a preparation stage, an initial orientation stage, and an improvement stage. In the preparation stage, the following is performed: measuring arrival times and signals amplitudes; estimating locations of the sensors 120; filtering outlier sensor-emitter pairs based on low signal intensity (e.g., below a threshold) or characteristics of going through too much non-soft tissue, i.e., over a predetermined threshold of a non-soft tissue; and, as an initial state, pointing each sensor 120 towards a center mass location of the sensors 120 at the height level of the sensor 120.

In the initial orientation stage, the following is performed: using a gradient-based optimization (for example, but not by way of limitation, using stochastic gradient descent SGD) algorithm, Broyden-Fletcher-Goldfarb-Shanoo (BFGS) algorithm, L-BFGS which is used for limited memory applications, adaptive moment estimation (Adam) Adam-W (a weighted Adam algorithm), multistage stochastic variational approximation gradient (M-SVAG), ADAbelief, as well as like optimization algorithms including gradient-based optimization algorithms) and derivatives thereof to fit the initial orientations based on a loss function, for example as described herein; and, optionally, adding a global maximization; and, measuring the loss, if loss improved return to the beginning of the initial orientation stage, otherwise, continue with the improvement stage. Adding the global maximization may include, but is not limited to: sorting the sensors 120 based on the loss value; selecting the highest loss's sensor (e.g., based on a predetermined threshold value), and changing the direction to the maximal intensity for selected sensors.

Finally, in the improvement stage, the following is performed: recalculating the location and orientation of sensors and emitters; and, performing an improvement loop. The improvement loop may include, but is not limited to: using FWI to improve the model based on the new location and new orientation of each sensor 120 and emitter 130; and, improving orientation based on the model. Repetition ceases once a predetermined threshold of improvement is reached.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for orientation determination, comprising:
causing emitters of a multi-sensor imaging apparatus to emit a first set of ultrasound signals, wherein the multi-sensor imaging apparatus includes the emitters and sensors;
measuring arrival times and amplitudes of the first set of ultrasound signals;
estimating initial locations of the sensors based on the measured arrival times and amplitudes;
pointing each of the sensors towards a center mass location;
fitting an initial orientation to each of the sensors and to each of the emitters using a loss function;
determining a coarse model based on the initial locations of the sensors and initial locations of the emitters;
calculating a new location and a new orientation for each of the sensors and each of the emitters based on the coarse model and a second set of ultrasound signals;
employing a full wave inversion (FWI) to generate an updated model based on the new locations and the new orientations of the sensors and emitters; and
determining an orientation of each of the sensors and each of the emitters based on the updated model.

2. The method of claim 1, wherein the at least one outlier sensor-emitter pair is filtered based on signal intensity, wherein each outlier sensor-emitter pair has a signal intensity below a threshold.

3. The method of claim 1, wherein the at least one outlier sensor-emitter pair is filtered based on a characteristic of signals passing through a non-soft tissue that is larger than a predetermined threshold size.

4. The method of claim 1, wherein fitting the initial orientation to each of the sensors and to each of the emitters further comprises:
performing a gradient descent.

5. The method of claim 4, wherein the gradient descent is based on any of: stochastic gradient descent (SGD), Broyden-Fletcher-Goldfarb-Shanoo, limited-memory Broyden-Fletcher-Goldfarb-Shanoo, adaptive moment estimation (ADAM), ADAM-W, multistage stochastic variational approximation gradient (M-SVAG), and ADAbelief.

6. The method of claim 1, further comprising:
filtering out at least one outlier sensor-emitter pair based on the estimated initial locations of the sensors.

7. The method of claim 6, wherein adding the global maximization further comprises:
sorting the sensors based on a loss value determined for each sensor using the loss function;
selecting at least one sensor of the sensors for which the determined loss value is above a predetermined threshold value;
changing a direction of each of the selected at least one sensor to a direction of maximal intensity; and
repeating a process of the sorting the sensors, the selecting at least one sensor, and the changing direction of each selected sensor until a loss value of each sensor is below the predetermined threshold value.

8. The method of claim 1, wherein determining the coarse model further comprises:
using an inversion tomography.

9. The method of claim 1, wherein the FWI is employed and the orientation is determined iteratively until a model generated based on the orientation converges.

10. A non-transitory computer readable medium having stored thereon instructions for causing a processing circuitry to execute a process, the process comprising:
causing emitters of a multi-sensor imaging apparatus to emit a first set of ultrasound signals, wherein the multi-sensor imaging apparatus includes the emitters and sensors;
measuring arrival times of the ultrasound signals and amplitudes of the first set of ultrasound signals;
estimating initial locations of the sensors based on the measured arrival times and amplitudes;
pointing each of the sensors towards a center mass location;
fitting an initial orientation to each of the sensors and to each of the emitters using a loss function;
determining a coarse model based on the initial locations of the sensors and initial locations of the emitters;
calculating a new location and a new orientation for each of the sensors and each of the emitters based on the coarse model and a second set of ultrasound signals;
employing a full wave inversion (FWI) to generate an updated model based on the new locations and the new orientations of the sensors and emitters; and
determining an orientation of each of the sensors and each of the emitters based on the updated model.

11. The non-transitory computer readable medium of claim 10, wherein the process further comprising:
filtering out at least one outlier sensor-emitter pair based on the estimated initial locations of the sensors.

12. A system, comprising:
a processing circuitry;
a plurality of emitters communicatively connected to the processing circuitry;
a plurality of sensors communicatively connected to the processing circuitry; and
a memory, the memory containing instructions that, when executed by the processing circuitry, configure the system to:
cause the plurality of emitters to emit a first set of ultrasound signals;
measure arrival times and amplitudes of the first set of ultrasound signals;
estimate initial locations of the sensors based on the measured arrival times and amplitudes;
point each of the plurality of sensors towards a center mass location;
fit an initial orientation to each of the plurality of sensors and to each of the plurality of emitters using a loss function;
determine a coarse model based on the initial locations of the plurality of sensors and initial locations of the plurality of emitters;
calculate a new location and a new orientation for each of the plurality of sensors and each of the plurality of emitters based on the coarse model and a second set of ultrasound signals;
employ a full wave inversion (FWI) to generate an updated model based on the new locations and the new orientations of the plurality of sensors and the plurality of emitters; and
determine an orientation of each of the plurality of sensors and each of the plurality of emitters based on the updated model.

13. The system of claim 12, wherein the at least one outlier sensor-emitter pair is filtered based on signal intensity, wherein each outlier sensor-emitter pair has a signal intensity below a threshold.

14. The system of claim 12, wherein at least one outlier sensor-emitter pair is filtered based on a characteristic of signals passing through a non-soft tissue that is larger than a predetermined threshold size.

15. The system of claim 12, wherein the system is further configured to:
perform a gradient descent.

16. The system of claim 15, wherein the gradient descent is based on any of: stochastic gradient descent (SGD), Broyden-Fletcher-Goldfarb-Shanoo, limited-memory Broyden-Fletcher-Goldfarb-Shanoo, adaptive moment estimation (ADAM), ADAM-W, multistage stochastic variational approximation gradient (M-SVAG), and ADAbelief.

17. The system of claim 12, wherein the system is further configured to:
filtering out at least one outlier sensor-emitter pair based on the estimated initial locations of the sensors.

18. The system of claim 17, wherein the system is further configured to:
sort the plurality of sensors based on a loss value determined for each sensor using the loss function;
select at least one sensor of the plurality of sensors for which the determined loss value is above a predetermined threshold value;
change a direction of each of the selected at least one sensor to a direction of maximal intensity; and
repeat a process of the sorting the plurality of sensors, the selecting at least one sensor, and the changing direction of each selected sensor until a loss value of each sensor is below the predetermined threshold value.

19. The system of claim 12, wherein the system is further configured to:
use an inversion tomography.

20. The system of claim 12, wherein the FWI is employed and the orientation is determined iteratively until a model generated based on the orientation converges.

21. The system of claim 18, wherein the system is further configured to:
determine a loss value for each sensor based on a full amplitude over all frequencies comparison between an observed measurement and a calculated signal for the sensor.

22. The system of claim 18, wherein the system is further configured to:
determine a loss value for each sensor based on a ratio of amplitudes at different frequencies.
